(19) United States
(12) Patent Application Publication
(10) Pub. No.: US 2022/0356499 A1
(43) Pub. Date: Nov. 10, 2022

(54) ENDOGLUCANASE, AND USE THEREOF

(71) Applicant: OZEKI CORPORATION, Nishinomiya-shi, Hyogo (JP)

(72) Inventors: Hironori SEMBA, Nishinomiya-shi, Hyogo (JP); Hirokazu TSUBOI, Nishinomiya-shi, Hyogo (JP); Takayuki BOGAKI, Nishinomiya-shi, Hyogo (JP); Akio KODA, Nishinomiya-shi, Hyogo (JP); Hiroyuki YAMADA, Nishinomiya-shi, Hyogo (JP); Kazuhiko ISHIKAWA, Ikeda-shi, Osaka (JP)

(21) Appl. No.: 17/621,992

(22) PCT Filed: Jul. 20, 2020
(86) PCT No.: PCT/JP2020/028039

§ 371 Date: Dec. 22, 2021

(30) Foreign Application Priority Data
Jul. 23, 2019 (JP) 2019-135102

(51) Int. Cl. C12P 19/12; C12N 9/42; C12P 19/14; C12N 1/20; C12N 15/70

(52) U.S. Cl. CPC C12P 19/12; C12N 9/2437; C12Y 302/01004; C12P 19/14; C12N 1/20; C12N 15/70; C12N 2800/101

(57) ABSTRACT

The present invention provides an endoglucanase having excellent heat resistance. Specifically, the present invention provides an endoglucanase satisfying characteristics (A) and (B) below:
(A) having an amino acid sequence that is at least 80% identical to the amino acid sequence of SEQ ID NO: 1; and
(B) having at least one amino acid substitution selected from the group consisting of K214E, D254E, and S309P.

### TECHNICAL FIELD

The present invention relates to technology involving endoglucanases.

### BACKGROUND ART

Although various techniques for saccharifying cellulose are available, the enzymatic saccharification technique, which requires less energy but produces a high yield of sugar, has been in the mainstream of development. Cellulase, which is a cellulose-degrading enzyme, is broadly divided into cellobiohydrolases, which act on the crystalline regions of cellulose, and endoglucanases, which act inside the cellulose molecular chain to reduce the molecular weight. β-glucosidase acts on a hydrosoluble oligosaccharide or cellobiose to catalyze the hydrolysis of their β-glycosidic bonds.

Endoglucanase (endo-β-1,4-glucanase (EC3.2.1.4)) is an effective enzyme for hydrolytic treatment of cellulose because it hydrolyzes β-1,4-glycosidic bonds between D-glucose, which is a constituent of cellulose. Endoglucanase catalyzes a reaction of endohydrolysis of β-1,4-bonds in not only cellulose, but also cellulose derivatives such as carboxymethylcellulose and hydroxyethylcellulose, lignin, mixed β-1,3-glucans such as cereal β-D-glucans, xyloglucans, and other plant materials containing cellulose moieties.

### CITATION LIST

### Patent Literature

PTL 1: U.S. Pat. No. 4,228,073

### Non-Patent Literature

NPL 1: Kishishita et al., J Ind Microbiol Biotechnol, 2015 42:137-141

### SUMMARY OF INVENTION

### Technical Problem

Treatment of plant samples or fiber products using an endoglucanase at high temperature attains a higher hydrolysis efficiency. If the endoglucanase is not inactivated under high temperature, not only can cellulose be hydrolyzed under high temperature, but also foreign substances such as other enzymes can be inactivated and modified by high temperature conditions, so that an endoglucanase itself that is capable of obtaining a target product at high purity can be efficiently purified. Further, such a heat-resistant endoglucanase can be efficiently collected and recycled after use. Accordingly, one object of the present invention is to provide an endoglucanase with high heat resistance.

### Solution to Problem

The following describes typical embodiments of the invention.

### Item 1.

An endoglucanase satisfying characteristics (A) and (B) below:

(A) having an amino acid sequence that is at least 80% identical to the amino acid sequence of SEQ ID NO: 1; and
(B) having at least one amino acid substitution selected from the group consisting of K214E, D254E, and S309P.

### Item 2.

The endoglucanase according to Item 1, having endoglucanase activity after heat treatment at 100° C. for 30 minutes.

### Item 3.

The endoglucanase according to Item 1 or 2, wherein the endoglucanase has the 173^{rd}, 271^{st}, and 314^{th} amino acid residues of the amino acid sequence of SEQ ID NO: 1.

### Item 4.

A DNA encoding the endoglucanase according to any one of Items 1 to 3.

### Item 5.

An expression vector incorporating the DNA according to Item 4.

### Item 6.

A transformant obtained by transformation with the vector according to Item 5.

### Item 7.

A method for producing the endoglucanase according to any one of Items 1 to 3, comprising the step of culturing the transformant according to Item 6.

### Item 8.

A method for producing a reducing sugar, comprising the step of reacting the endoglucanase according to any one of Items 1 to 3 with a sample containing cellulose at 70° C. or more.

### Item 9.

A method for separating an endoglucanase, comprising the step of treating the endoglucanase according to any one of Items 1 to 3 at 80° C. or more.

### Advantageous Effects of Invention

The present invention provides an endoglucanase with high heat resistance. In one preferable embodiment, a means for efficiently producing a reducing sugar from cellulose is provided. In one preferable embodiment, a means for efficiently separating an endoglucanase is provided.

### BRIEF DESCRIPTION OF DRAWINGS

FIG. 1 shows the thermal stability of mutant EGPh expressed in Aspergillus niger. Error bars indicate the standard error. “**” represents p<0.01 (vs. wild type), and “N.D.” represents not detected.

FIG. 2 shows the thermal stability of mutant EGPh expressed in Escherichia coli. Error bars indicate the standard error. “**” represents p<0.01 (vs. wild type), and “*” represents p<0.05 (vs. wild type).

### DESCRIPTION OF EMBODIMENTS

### 1. Endoglucanase

The endoglucanase preferably has the following characteristics (A) and (B):

(A) having an amino acid sequence that is at least 80% identical to the amino acid sequence of SEQ ID NO: 1; and
(B) having at least one amino acid substitution selected from the group consisting of K214E, D254E, and S309P.

The amino acid sequence represented by SEQ ID NO: 1 is an amino acid sequence (not including a signal peptide) comprising a wild-type endoglucanase from hyperthermophilic archaeon Pyrococcus horikoshii.

The endoglucanase preferably has (A) an amino acid sequence that is at least 80% identical to the amino acid sequence of SEQ ID NO: 1, and (B) at least one amino acid substitution selected from the group consisting of K214E, D254E, and S309P. This is presumably because by the mutation, an amino acid exposed on the surface of an enzyme that is easily affected by heat is substituted, which results in attaining a thermally stable three-dimensional structure. Regarding the codes that represent each type of substitution (B), the number indicates the position of an amino acid in the amino acid sequence of SEQ ID NO: 1. The letter before the number indicates the type of the amino acid originally present at the position. The letter after the number indicates the type of the amino acid that substitutes the original amino acid. For example, “K214E” means that lysine (K) at position 214 in the amino acid sequence of SEQ ID NO: 1 is substituted with glutaminic acid (E). The other codes representing substitution are interpreted in the same manner.

The identity with the amino acid sequence of SEQ ID NO: 1 in (A) is preferably 80% or more, 85% or more, 90% or more, 91% or more, 92% or more, 93% or more, 94% or more, 95% or more, 96% or more, 97% or more, 98% or more, or 99% or more. It is more preferably 95% or more, even more preferably 98% or more, and particularly preferably 99% or more.

The amino acid sequence identity can be determined by using a commercially available analytical tool or an analytical tool available through telecommunication lines (Internet). For example, the amino acid sequence identity can be determined by using ClustalW Ver. 2.1 Pairwise Alignment (http://clustalw.ddbj.nig.ac.jp/index.php?lang=ja) with default parameters (default setting). Alternatively, the amino acid sequence identity can be determined by using the Basic Local Alignment Search Tool (BLAST) (http://www.ncbi.nlm.nih.gov/BLAST/) available from the National Center for Biotechnology Information (NCBI) with default parameters.

The addition of one or more amino acid substitutions to a specific amino acid sequence is a well-known technique in the art, and any technique can be used. Such a substitution can be made by using, for example, a restriction enzyme treatment, treatment using an exonuclease, DNA ligase, etc., site-directed mutagenesis, or random mutagenesis.

In the specific amino acid substitution (B), only one type of substitution may be added to the amino acid sequence of SEQ ID NO: 1 or an amino acid sequence that is at least 80% identical to the amino acid sequence of SEQ ID NO: 1. A combination of two or more types of substitution may be added to the amino acid sequence of SEQ ID NO: 1 or an amino acid sequence that is at least 80% identical to the amino acid sequence of SEQ ID NO: 1. When only one type of amino acid substitution is added, the substitution is preferably K214E or S309P, and more preferably S309P. When a combination of two or more types of amino acid substitution are added to the amino acid sequence of SEQ ID NO: 1 or an amino acid sequence that is at least 80% identical to the amino acid sequence of SEQ ID NO: 1, the combination can be any combination. The combination may be K214E and D254E, K214E and S309P, D254E and S309P, or K214E, D254E, and S309P

The endoglucanase activity can be measured by any technique; however, in this specification, it is measured by the Nelson-Somogyi method unless otherwise specified. Specifically, 200 μl of a 50 mM sodium acetate buffer (pH of 5.0) containing a carboxymethylcellulose sodium salt with a final concentration of 1 wt % as a substrate is prepared, a specific amount of endoglucanase is added to the buffer to start the reaction, and the amount of reducing sugar produced at 70° C. for 10 min is quantified. By defining the amount of enzyme that releases a reducing sugar in an amount equivalent to 1 μmol of glucose per minute as 1 U, endoglucanase activity per unit weight can be measured.

In one embodiment, the endoglucanase preferably has endoglucanase activity after heat treatment at 100° C. for 30 minutes. In one embodiment, the endoglucanase preferably has an endoglucanase activity (residual activity), which is measured by the Nelson-Somogyi method after heat treatment at 100° C. for 30 minutes relative to the case without heat treatment, of 5% or more, 8% or more, 10% or more, 20% or more, 30% or more, or 35% or more.

In one embodiment, the endoglucanase preferably has an endoglucanase activity (residual activity), which is measured by the Nelson-Somogyi method after heat treatment at 98° C. for 30 minutes relative to the case without heat treatment, of 30% or more, 40% or more, 50% or more, or 60% or more. In one embodiment, the endoglucanase preferably has an endoglucanase activity, which is measured by the Nelson-Somogyi method after heat treatment at 98° C. for 30 minutes relative to the case without heat treatment, 1.2 times or more, 1.4 times or more, 1.6 times or more, or 2 times or more as high as the residual activity of the wild type.

The heat treatment can be performed by adding the endoglucanase in an amount of 1 U/ml to a sodium phosphate buffer (pH of 7.0) having a final concentration of 200 mM, dissolving or suspending the resultant, and keeping the resultant for a predetermined period (e.g., 30 min.) in a thermostatic bath, which has been set to a predetermined temperature.

From the viewpoint that the higher-order structure, phenotype, or properties of the endoglucanase having the amino acid sequence of SEQ ID NO: 1 are not adversely affected in a significant manner, it is preferable to conserve the 173rd, 271st, and 314th amino acid residues of the amino acid sequence of SEQ ID NO: 1. These amino acid residues are considered to correspond to the active center of endoglucanase. It is also preferable to conserve the 41st, 44th, 74th, 127th, 128th, 172nd, 245th, 269th, 349th, and 357th amino acid residues of the amino acid sequence of SEQ ID NO: 1. These amino acid residues are considered to involve the binding of the substrate of endoglucanase.

The endoglucanase described above can be produced by a genetic engineering technique using DNA as described below. The endoglucanase can also be produced using a general protein chemical synthesis method (e.g., liquid-phase and solid-phase methods) based on information on the amino acid sequence represented by SEQ ID NO: 1.

### 2. DNA Encoding Endoglucanase

The base sequence of a DNA encoding the endoglucanase is not particularly limited. In one embodiment, the DNA preferably has a base sequence with the specific degree of identity with the base sequence of SEQ ID NO: 2. The specific degree of identity refers to, for example, 60% or more, 70% or more, 80% or more, 90% or more, 95% or more, 96% or more, 97% or more, 98% or more, or 99% or more. SEQ ID NO: 2 is the base sequence encoding the amino acid sequence of SEQ ID NO: 1.

The identity of the base sequence can be determined by using a commercially available analytical tool, or an analytical tool available through telecommunication lines (Internet). For example, software such as FASTA, BLAST, PSI-BLAST, or SSEARCH can be used to determine the identity. The major initial conditions typically applied to a BLAST search are specifically as follows. In Advanced BLAST 2.1, a blastn program is used, and the parameters are set to default values to perform a search, thus calculating the identity value (%) of a nucleotide sequence.

In one embodiment, the DNA is preferably present in an isolated state. As used herein, “DNA in an isolated state” means that the DNA is separated from components such as other nucleic acids and proteins that naturally accompany it. However, the DNA may contain a portion of other nucleic acid components, such as nucleic acid sequences that naturally flank the DNA sequence (e.g., the promoter region sequence and terminator sequence). DNAs prepared by a genetic engineering technique, such as cDNA molecules, are, when in an isolated state, preferably substantially free of other components such as cell components and culture media. Likewise, in DNAs prepared by a chemical synthesis, “DNA in an isolated state” preferably means that the DNA is substantially free of precursors (starting materials) such as dNTP, as well as chemical substances, etc., used in the synthetic process.

The DNA can easily be obtained on the basis of the base sequence of SEQ ID NO: 2 by using a chemical DNA synthesis method (e.g., phosphoramidite method) or a genetic engineering technique.

### 3. Vector

The vector preferably includes the DNA in an expressible manner. The type of the vector is suitably selected according to the type of the host cell. Examples of vectors include plasmid vectors, cosmid vectors, phage vectors, and virus vectors (e.g., adenoviral vectors, retroviral vectors, and herpes viral vectors).

Examples of vectors that enable expression in Escherichia coli include pUC19, pUC18, pBR322, pHSG299, pHSG298, pHSG399, pHSG398, RSF1010, pMW119, pMWll8, pMW219, pMW218, pQE, and pET. Examples of vectors that enable expression in yeast include pBR322, pJDB207, pSH15, pSH19, pYepSecl, pMFa, pYES2, pHIL, pPIC, pA0815, pPink. Examples of vectors that enable expression in insects include pAc, pVL, and pFastbac.

For a eukaryotic host cell, usable expression vectors include those comprising, at the upstream of the polynucleotide to be expressed, a promoter, an RNA splicing site, a polyadenylation site, a transcription termination sequence, and the like. The expression vectors may further optionally comprise a replication origin, a secretion signal, an enhancer, and/or a selection marker.

### 4. Transformant

The transformant is preferably transformed with the above vector. In the transformant, the vector may be present autonomously in the host cell or incorporated into the genome in a homologous or non-homologous recombination manner. The host cell for use in transformation is not particularly limited as long as the endoglucanase can be produced, and either prokaryotic cells or eukaryotic cells can be used. Specific examples of host cells include prokaryotic cells including bacteria of genus Escherichia coli such as Escherichia coli (e.g., HB101, MC1061, JM109, CJ236, and MV1184), coryneform bacteria such as Corynebacterium glutamicum, actinomycetes such as bacteria of genus Streptomyces, bacteria of genus Bacillus such as Bacillus subtilis, bacteria of genus Streptococcus, and bacteria of genus Staphylococcus; yeast such as genus Saccharomyces, genus Pichia, and genus Kluyveromyces, and fungal cells such as genus Aspergillus, genus Penicillium, genus Talaromyces, genus Trichoderma, genus Hypocrea, and genus Acremonium; insect cells including Drosophila S2, Spodoptera Sf9, and silkworm-culturing cells; and plant cells. It is also possible to produce the endoglucanase in a medium by exploiting the protein secretion capacity of Bacillus subtilis, yeast, fungus, actinomycetes, and the like.

To introduce a recombinant expression vector into a host cell, a conventional method can be used. Examples include a variety of methods such as a competent cell method, a protoplast method, an electroporation method, a microinjection method, and a liposome fusion method. However, the method is not limited to these.

The transformant is capable of producing an endoglucanase, and thus can be used for producing the endoglucanase. The transformant itself can also be used for producing reducing sugars, such as glucose, cellobiose, and cello-oligosaccharides from samples containing cellulose.

### 5. Production Method of Endoglucanase Using Transformant

The Endoglucanase can be produced by culturing the transformant and collecting the endoglucanase from the cultured product. The culture can be performed using a passage culture or batch culture with a medium suitable for the host cell. The culture can be performed until a sufficient amount of the endoglucanase is produced, with monitoring the activity of the endoglucanase produced inside and outside of the transformant as a guide.

The culture medium may be suitably selected from conventionally used media according to the type of the host cell. The culture can be performed under conditions suitable for growth of the host cell. Examples of media used for culturing Escherichia coli include nutrient media such as LB medium, and minimal media to which a carbon source, a nitrogen source, a vitamin source, and the like are added, such as M9 medium.

The culture conditions can be suitably determined according to the type of the host cell. The culture is typically performed at 16 to 42° C., preferably 25 to 37° C., for 5 to 168 hours, preferably for 8 to 72 hours. Depending on the host, either shaking culture or static culture can be used, and agitation and/or ventilation may optionally be provided. When an induction promoter is used for gene expression, a promoter-inducing agent may be added to the medium to perform a culture.

Purification or isolation of the endoglucanase from the cultured supernatant can be performed by suitably combining known techniques. Examples of techniques for use include ammonium sulfate precipitation, solvent precipitation (e.g., ethanol), dialysis, ultrafiltration, acid extraction, and a variety of chromatographic approaches (e.g., gel filtration chromatography, anion- or cation-exchange chromatography, phosphocellulose chromatography, hydrophobic interaction chromatography, affinity chromatography, hydroxyapatite chromatography, lectin chromatography, and high-performance liquid chromatography). Examples of carriers used in affinity chromatography include carriers to which an antibody against the endoglucanase is bound and carriers to which a substance with affinity for a peptide tag is bound when the peptide tag is added to the endoglucanase.

When the endoglucanase is accumulated inside the host cells, the transformed cells are disrupted, and the endoglucanase is purified or isolated from the centrifuged supernatant of the disrupted product by the techniques described above. For example, after completion of culture, the cells collected by centrifugation are suspended in a buffer for cell disruption (20 to 100 mM Tris-HCl (pH of 8.0), 5 mM EDTA) and disrupted by ultrasonication. The disruption-treated fluid is centrifuged at 10000 to 15000 rpm for 10 to 15 minutes to thereby obtain a supernatant. The precipitate obtained after centrifugation can optionally be solubilized with guanidinium chloride, urea, or the like, and then further purified.

### 6. Production Method of Reducing Sugars Using Endoglucanase

By the reaction of the endoglucanase with a sample containing cellulose (e.g., a biomass resource), the cellulose is decomposed to produce molasses containing a reducing sugar. Examples of the reducing sugar include glucose, cellobiose, cello-oligosaccharides, and the like. When a biomass resource is used as a sample containing cellulose, it is preferable to use other enzymes such as cellulase in combination with the endoglucanase to produce molasses more efficiently.

The type of the sample containing cellulose is not particularly limited as long as the sample can be decomposed by the endoglucanase of the present invention. Examples of the sample containing cellulose include bagasse, wood, bran, wheat straw, pasture grasses of Gramineae or Papilionaceae, corncobs, bamboo grass, pulp, rice straw, chaff, wheat bran, soybean meal, soy pulp, coffee grounds, and rice bran.

The temperature at which the endoglucanase is reacted with a sample containing cellulose is preferably 70° C. or more, 75° C. or more, 80° C. or more, 85° C. or more, 90° C. or more, 95° C. or more, or 98° C. or more.

Molasses containing a reducing sugar can be produced from a sample containing cellulose according to a known technique. Biomass resources for use may be either dried materials or wet materials. The materials are preferably milled into particles of 100 to 10000 μm in size beforehand to increase processing efficiency. Milling is performed by using a device such as a ball mill, a vibrational mill, a cutter mill, or a hammer mill. The milled biomass resource is immersed in water, steam, or an alkaline solution, and subjected to a high temperature treatment or a high temperature high pressure treatment at 60 to 200° C. to further increase the enzymatic treatment efficiency. For example, alkali treatment can be performed using caustic soda, ammonia, or the like. The biomass sample that has been subjected to such a pretreatment is suspended in an aqueous vehicle, and the endoglucanase and cellulase are added thereto, followed by heating with stirring to thereby decompose or saccharize the biomass resource.

When the endoglucanase is reacted with a sample containing cellulose in an aqueous solution, the pH and other conditions in the reaction solution may be within the range in which the endoglucanase is not inactivated.

The molasses containing a reducing sugar may be used unmodified, or may be used as a dry product after removing water. It is also possible to further isomerize or decompose the molasses by a chemical reaction or enzymatic reaction depending on the intended use. The molasses or its fraction can be used, for example, as a starting material for alcohols such as methanol, ethanol, propanol, isopropanol, butanol, and butanediol by a fermentation process.

### 7. Method for Separating Endoglucanase

In the purification of endoglucanase, the endoglucanase-containing sample can be treated at 80° C. or more, thereby inactivating foreign proteins to obtain an endoglucanase with high purity. Further, by treating at 80° C. or more a solution containing foreign substances (e.g., other enzymes or microorganisms) in addition to the endoglucanase, such as a solution obtained after the endoglucanase is reacted with the sample containing cellulose, the foreign enzymes and microorganisms can be inactivated while maintaining the activity of endoglucanase. In one embodiment, the processing temperature can be 80° C. or more, 85° C. or more, 90° C. or more, 95° C. or more, 98° C. or more, or 100° C. or more. The treatment time may be within the range in which the endoglucanase is not inactivated.

The method for separating the endoglucanase can be performed according to a known technique. For example, the endoglucanase and foreign substance can be separated by filtration, centrifugation, microfiltration, rotary vacuum filtration, ultrafiltration, pressurized filtration, cross membrane microfiltration, cross-flow membrane microfiltration, or similar methods.

### EXAMPLES

The invention is described in more detail using the Examples below, but the invention is not limited to these.

### 1. Construction of Wild-Type Endoglucanase Expression Vector

The endoglucanase gene described in SEQ ID NO: 2 was synthesized and inserted in a plasmid pSENSU (Takaya, T. et al. Appl Microbiol Biotechnol (2011) 90: 1171.) that had been previously produced by the inventors. This plasmid contains a secretion signal derived from an α-amylase gene, and can introduce the target gene between the secretion signal and a terminator by a PmlI-XbaI treatment.

The endoglucanase gene described in SEQ ID NO: 2 was introduced into the pSENSU vector according to the following procedure. After the PmlI-XbaI digestion of pSENSU, the pSENSU was subjected to agarose gel electrophoresis to isolate and purify the pSENSU-PmlI-XbaI digested fragment. Using the synthesized endoglucanase gene described in SEQ ID NO: 2 as a template, insertion fragments were amplified by the PCR method using the primers of SEQ ID NOs: 3 and 4. The amplified fragments were digested with XbaI, and subjected to agarose gel electrophoresis, followed by isolation and purification. The obtained endoglucanase gene was ligated into the PmlI-XbaI site of pSENSU, thus constructing an endoglucanase expression vector pSENSU-EGPh.

### 2. Construction of Endoglucanase Having Amino Acid Substitution Expression Vector

As compared to the basic sequence shown in SEQ ID NO: 2, V25P has an amino acid substitution in which valine at position 25 is substituted with proline, H48Y has an amino acid substitution in which histidine at position 48 is substituted with tyrosine, Q87M has an amino acid substitution in which glutamine at position 87 is substituted with methionine, H133F is an amino acid substitution in which histidine at position 133 is substituted with phenylalanine, K214E is an amino acid substitution in which lysine at position 214 is substituted with glutamic acid, D254E has an amino acid substitution in which aspartic acid at position 254 is substituted with glutamic acid, and S309P has an amino acid substitution in which serine at position 309 is substituted with proline. Endoglucanase genes having such amino acid substitutions were synthesized, and pSENSU-EGPh_V25P, pSENSU-EGPh_H48Y, pSENSU-EGPh_Q87M, pSENSU-EGPh_H133F, pSENSU-EGPh_K214E, pSENSU-EGPh_D254E, and pSENSU-EGPh_S309P were constructed according to the same method as that of the wild type.

### 3. Obtainment of Transformant and Endoglucanase

Using pSENSU-EGPh, pSENSU-EGPh_V25P, pSENSU-EGPh_H48Y, pSENSU-EGPh_Q87M, pSENSU-EGPh_H133F, pSENSU-EGPh_K214E, pSENSU-EGPh_D254E, or pSENSU-EGPh_S309P, Aspergillus niger NS48 strains (double destruction strain of niaD and sC obtained by mutation treatment) were transformed by a protoplast-PEG method. Genomic DNAs were extracted from the obtained transformants, thus obtaining transformants with one or more copies of the plasmid introduced therein by a real-time PCR method. These transformants were cultured in dextrin-peptone medium (4 wt % dextrin, 2 wt % polypeptone, 2 wt % yeast extract, 0.5 wt % KH₂PO₄, 0.05 wt % MgSO₄. 7H₂O) for 6 days, and the culture supernatants were used as crude enzyme solutions to measure endoglucanase activity. Specifically, the endoglucanase activity was measured as follows. A reaction was started by adding 10 μl of the crude enzyme solution to 200 μl of a 50 mM sodium acetate buffer (pH of 5.0) containing a carboxymethylcellulose sodium salt having a final concentration of 1 wt % as a substrate, and the amount of the reducing sugar generated at 70° C. for 10 minutes was determined by the Nelson-Somogyi method. The amount of the enzyme that releases a reducing sugar in an amount equivalent to 1 μmol of glucose per minute was defined as 1 U, and strains having an endoglucanase activity of 0.1 U or more per ml of a crude enzyme solution were obtained as endoglucanase-producing strains.

### 4. Comparison of Thermal Stability

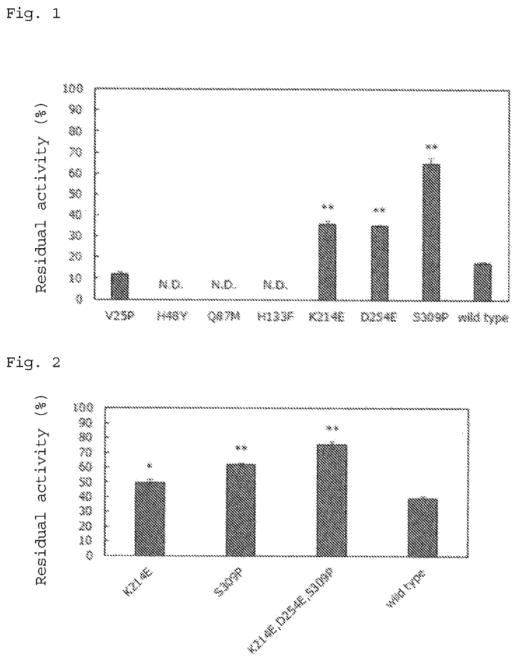

Each of the crude enzyme solutions obtained above was heated at 80° C. for 30 minutes, and then centrifuged at 13,000 rpm for 5 minutes. The supernatant was used as a crude purified enzyme solution. The crude purified enzyme solution was heated in a heat block at 98° C. for 30 minutes and centrifuged at 13,000 rpm for 5 minutes. Thereafter, the endoglucanase activity measurement was performed on the supernatant by the method described in Item 3 above. As a control, the activity of the unheated sample was measured in the same manner, and the residual activity after heating was calculated as a relative value. The experiment was conducted in triplicate, and the mean value and standard error were calculated. The results are shown in FIG. 1.

H48Y, Q87M, and H133F showed no residual activity. V25P showed lower residual activity than that of the wild type; however, K214E, D254E, and S309P showed a significant improvement in thermal stability. In particular, S309P had a residual activity three times or more as high as that of the wild type.

When the crude purified enzyme solution was heated at 100° C. for 30 minutes, the endoglucanase activity of the wild type disappeared while the endoglucanase activity of K214E, D254E, and S309P remained.

5. Obtainment of E. coli Transformant and Endoglucanase

K214E, D254E, and S309P respectively have an amino acid substitution in which lysine at position 214 is substituted with glutamic acid, an amino acid substitution in which aspartic acid at position 254 is substituted with glutamic acid, and an amino acid substitution in which serine at position 309 is substituted with proline. Expression vectors of endoglucanase gene having an amino acid substitution such as K214E or S309P, or amino acid substitutions such as K214E, D254E, and S309P were constructed by a conventional method, and crude enzyme solutions were extracted from endoglucanase-producing strains obtained by transformation of E. coli BL21 (DE3) strains, thus measuring the endoglucanase activity according to the method described in Item 4 above. The results are shown in FIG. 2. As shown in FIG. 2, the thermal stability was improved by the introduction of amino acid substitution as compared to the wild type.

The results indicate that the introduction of at least one amino acid substitution selected from the group consisting of K214E, D254E, and S309P improves the thermal stability of endoglucanase.

Sequence Listing

1. An endoglucanase satisfying characteristics (A) and (B) below:
(A) having an amino acid sequence that is at least 80% identical to the amino acid sequence of SEQ ID NO: 1; and
(B) having at least one amino acid substitution selected from the group consisting of K214E, D254E, and S309P.

2. The endoglucanase according to claim 1, having endoglucanase activity after heat treatment at 100° C. for 30 minutes.

3. The endoglucanase according to claim 1, wherein the endoglucanase has the 173^{rd}, 271^{st}, and 314^{th} amino acid residues of the amino acid sequence of SEQ ID NO: 1.

4. A DNA encoding the endoglucanase according to claim 1.

5. An expression vector incorporating the DNA according to claim 4.

6. A transformant obtained by transformation with the vector according to claim 5.

7. A method for producing an endoglucanase, comprising the step of culturing the transformant according to claim 6.

8. A method for producing a reducing sugar, comprising the step of reacting the endoglucanase according to claim 1 with a sample containing cellulose at 70° C. or more.

9. A method for separating an endoglucanase, comprising the step of treating the endoglucanase according to claim 1 at 80° C. or more.

10. The endoglucanase according to claim 3, having endoglucanase activity after heat treatment at 100° C. for 30 minutes.

11. A DNA encoding the endoglucanase according to claim 3.

12. An expression vector incorporating the DNA according to claim 11.

13. A transformant obtained by transformation with the expression vector according to claim 12.

14. A method for producing an endoglucanase, comprising the step of culturing the transformant according to claim 13.

15. A method for producing a reducing sugar, comprising the step of reacting the endoglucanase according to claim 3 with a sample containing cellulose at 70° C. or more.

16. A method for separating an endoglucanase, comprising the step of treating the endoglucanase according to claim 3 at 80° C. or more.


SEQUENCE LISTING (4 sequences)

SEQ ID NO: 1 - protein, 388 aa, Pyrococcus horikoshii
ENTTYQTPTGIYYEVRGDTIYMINVTSGEETPIHLFGVNWFGFETPNHVVHGLWKRNWEDMLLQIKSLGFNAIRLPFCTESVKPGTQPIGIDYSKNPDLRGLDSLQIMEKIIKKAGDLGIFVLLDYHRIGCTHIEPLWYTEDFSEEDFINTWIEVAKRFGKYWNVIGADLKNEPHSVTSPPAAYTDGTGATWGMGNPATDWNLAAERIGKAILKVAPHWLIFVEGTQFTNPKTDSSYKWGYNAWWGGNLMAVKDYPVNLPRNKLVYSPHVYGPDVYNQPYFGPAKGFPDNLPDIWYHHFGYVKLELGYSVVIGEFGGKYGHGGDPRDVIWQNKLVDWMIENKFCDFFYWSWNPDSGDTGGILQDDWTTIWEDKYNNLKRLMDSCSKSS

SEQ ID NO: 2 - DNA, 1167 bp, Pyrococcus horikoshii
gaaaatacaacatatcaaacaccgactggaatttactacgaagtgagaggagatacgatatacatgattaatgtcaccagtggagaggaaactcccattcatctctttggtgtaaactggtttggctttgaaacacctaatcatgtagtgcacggactttggaagagaaactgggaagacatgcttcttcagatcaaaagcttaggcttcaatgcaataagacttcctttctgtactgagtctgtaaaaccaggaacacaaccaattggaatagattacagtaaaaatccagatcttcgtggactagatagcctacagattatggaaaagatcataaagaaggccggagatcttggtatctttgtcttactcgactatcataggataggatgcactcacatagaacccctctggtacacggaagacttctcagaggaagactttattaacacatggatagaggttgccaaaaggttcggtaagtactggaacgtaataggggctgatctaaagaatgagcctcatagtgttacctcacccccagctgcttatacagatggtaccggggctacatggggtatgggaaaccctgcaaccgattggaacttggcggctgagaggataggaaaagcgattctgaaggttgcccctcattggttgatattcgtggaggggacacaatttactaatccgaagactgacagtagttacaaatggggctacaacgcttggtggggaggaaatctaatggccgtaaaggattatccagttaacttacctaggaataagctagtatacagccctcacgtatatgggccagatgtctataatcaaccgtactttggtcccgctaagggttttccggataatcttccagatatctggtatcaccactttggatacgtaaaattagaactaggatattcagttgtaataggagagtttggaggaaaatatgggcatggaggcgatccaagggatgttatatggcaaaataagctagttgattggatgatagagaataaattttgtgatttcttttactggagctggaatccagatagtggagataccggagggattctacaggatgattggacaacaatatgggaagataagtataataacctgaagagattgatggatagttgttccaaaagttcttag

SEQ ID NO: 3 - DNA, 31 bp, Artificial
  Primer
ctgctttggctgaaaatacaacatatcaaac

SEQ ID NO: 4 - DNA, 30 bp, Artificial
  Primer
gcattctagactaagaacttttggaacaac